(19) United States
(12) Patent Application Publication
(10) Pub. No.: US 2022/0008494 A1
(43) Pub. Date: Jan. 13, 2022

(54) COMPOSITION FOR USE IN THE PREVENTION AND/OR SYMPTOMATIC TREATMENT OF IRRITABLE BOWEL SYNDROME

(71) Applicant: NEILOS S.R.L., Piano di Sorrento (Napoli) (IT)

(72) Inventor: Umberto DI MAIO, Sant'Agnello (Napoli) (IT)

(21) Appl. No.: 17/295,679

(22) PCT Filed: Nov. 21, 2018
(86) PCT No.: PCT/IB2019/060029

§ 371 Date: May 20, 2021

(30) Foreign Application Priority Data
Nov. 21, 2018 (IT) 102018000010473

(51) Int. Cl. A61K 36/48; A61K 31/19

(52) U.S. Cl. CPC A61K 36/48; A61K 31/19

(57) ABSTRACT

A synergistic composition of active ingredients which is particularly effective in the treatment and/or prevention of irritable bowel syndrome is provided. The composition includes the synergistic combination of butyric acid or a salt thereof and Tamarindus indica extract.

The present invention relates to a composition of substances preferably obtained from natural sources, which is effective in the prevention and/or symptomatic treatment of irritable bowel syndrome.

Irritable bowel syndrome (IBS), or irritable colon syndrome, is a chronic gastrointestinal disorder, which is neither hereditary nor infectious, generally appearing between 15 and 40 years of age with an estimated frequency of 10-20%, and is more frequent in women than in men. It is a functional disorder not due to bowel anatomical changes, but to impaired activity of the bowel muscles. If the muscles contract too much, diarrhea occurs (IBS-D), if instead they contract too little, constipation occurs (IBS-C); if the muscles undergo prolonged contractions (spasms), pain occurs. Therefore, the disorder in question is characterised by abdominal pain, bowel irregularity, meteorism and variation in stool consistency with recurrent, chronic course, characterised by periods of exacerbation and periods of remission. The symptoms can be variously combined with each other. The causes of irritable bowel syndrome are not yet well defined. Visceral hypersensitivity is considered one of the main factors in its etiopathogenesis, particularly in those subjects who have, as the predominant symptom, abdominal pain resulting from abnormal release of chemical substances from the intestine (potassium, ATP, bradykinins, prostaglandin E2), which, in turn, result in the release of chemical mediators from nerve endings. The term “irritable”, in fact, is used to indicate that the nerves running within the intestine are abnormally sensitive and the nerves controlling the intestine muscles are excessively active. All this results in over-response of the intestine to everything that normally occurs, such as the passage of gas or fluids throughout its length. This leads to inappropriate intestinal muscle activity that, for example, can temporarily stop a bowel movement or frustrate the efforts to expel faeces. Therefore, the diagnosis of IBS is not simple: the most obvious signs are abdominal pain or discomfort, relieved by evacuation or associated with alternating bowel habit or variable stool consistency. Pain is accompanied by at least two of the following symptoms:
altered stool passage: straining, urgency, incomplete evacuation;
abdominal bloating, distention, tension or hard feeling in the abdomen;
worsening of symptoms at mealtimes;
presence of mucus.

Other symptoms are drowsiness, nausea, back pain and urinary disorders, which can be used to support the diagnosis. The most commonly used tests to confirm the diagnosis of IBS are ultrasound, sigmoidoscopy, colonoscopy, and barium enema. Accurate collection of clinical history is usually the key to the diagnosis of irritable bowel syndrome. To lead to a correct diagnosis, patients must have shown at least three of the following symptoms continuously and for at least 3 months within the last year:

1) Intestinal pain or disorder characterised by relief after evacuation;

2) Pain associated with the change in evacuation frequency;

3) Pain associated with the change in stool consistency.

These symptoms may be accompanied by abdominal distension and susceptibility to bloating, or changes in evacuation frequency. The latter symptoms may be present but are not fundamental symptomatic elements. It is important that the evaluation of these criteria is completed by ruling out other clinical pictures, which show similar symptoms and include organic diseases or other functional disorders. Irritable bowel syndrome in some individuals can disrupt the quality of life, meaning that it can significantly affect daily activities.

Effective treatment of this condition in milder cases comprises self-management with attention to nutrition and to a healthy lifestyle.

In more complicated cases, drug therapy is used instead.

Drug therapy is based on drugs that act by antagonizing the action of acetylcholine. Drugs that perform this type of action are therefore anticholinergic drugs or muscarinic receptor antagonists. Their functions also consist in inhibiting the hyperexcitability of the smooth muscles of the small intestine and colon. In particular, drug therapy encompasses the use of hyoscyamine (atropine) and dicyclomine (dicycloverine): both have a relaxant (spasmolytic) effect on smooth muscles.

However, at high doses, these drugs induce major anticholinergic-type adverse effects, including visual impairment, urinary retention and constipation, and for this reason they are not frequently used.

Sometimes drug therapy combines anticholinergic drugs with antidepressant drugs, such as imipramine and amitriptyline, which can be administered simultaneously. However, these drugs can also cause different types of side effects that may arise due to the stimulation of acetylcholine and histamine receptors, for example dizziness, weight gain, blurred vision, sedation, tremors, and even epilepsy and heart rhythm disorders.

Abnormal intestinal microflora, in particular a decrease in Bifidobacteria and Lactobacilli, has been observed in patients suffering from irritable bowel syndrome. This disruption of the intestinal ecosystem generates a state of dysbiosis which contributes to the onset of the typical symptoms of irritable bowel syndrome: gas production, change in motility, and increased sensitivity of the intestinal tract.

The availability of a product based on natural substances, which is able to rebalance the microenvironment and restore normal intestinal physiology, would therefore be extremely useful and advantageous for subjects suffering from irritable bowel syndrome. A product based on natural substances would also allow the avoidance of the aforementioned conventional treatments for irritable bowel syndrome, which have the adverse effects described above.

These and other needs are met by the present invention which provides a composition characterised in that it comprises a synergistic combination of active substances obtained from natural sources, the aforesaid combination having proved particularly effective in the symptomatic treatment of irritable bowel syndrome.

The composition of the invention is as defined in appended claim 1. Further features and advantages of the invention are defined in the dependent claims. The claims form an integral part of the present specification.

A detailed description of some preferred embodiments of the invention is provided hereinafter.

The synergistic composition of the present invention is useful for the treatment and prevention of irritable bowel syndrome.

In the composition of the present invention, the synergistic action takes place between butyric acid, or a salt thereof, and Tamarindus indica extract.

Butyric acid, also known as butanoic acid, is a carboxylic acid that is mainly found esterified with glycerol in many natural, animal and plant fats. Normal butyric acid, also referred to as fermentation butyric acid, is also found as a hexyl ester in the oil of Heracleum giganteum and as an octyl ester in Pastinaca saliva, which are plants belonging to the same family; it has also been found in living flesh, during sweating processes. Normally, butyric acid is a compound that is prepared from sugars or starch, through fermentation triggered by fermented cheeses, with the addition of calcium carbonate in order to salify other acids that may form in the process. Butyric fermentation of starch is aided by the direct addition of Bacillus subtilis. Butyric, acetic and propionic acid account for about 83% of the short-chain fatty acids present in the human colon. The concentration of these acids in the intestinal lumen ranges from 60 to 150 mmol/kg and they are in an acetate-propionate-butyrate ratio of 60:25:10. Short-chain fatty acids are rapidly absorbed by the epithelium of the gastrointestinal tract. In the large intestine, absorption reaches the highest peaks in the cecum and the ascending colon through both active and passive transport, whereas the levels of butyric acid production in the sigmoid colon and the rectum are low. Butyrate is the preferred energy source for colon epithelial cells, while a well-balanced diet, rich in fibre, probiotics and prebiotics, is the preferred source of butyrate. Similarly to other short-chain fatty acids (acetic and propionic acid), endogenous butyric acid is produced through bacterial fermentation of non-digestible carbohydrates and hexose oligomers with different degrees of polymerization, such as non-starch polysaccharides, resistant (particularly butyrogenic) starches, oligosaccharides (inulin and FOS), disaccharides (lactose), and alcohol sugars (mannitol and sorbitol). The bacterial species involved in the production of butyrate are Clostridium spp., Eubacterium spp., Fusobacterium spp., Butyrivibrio spp., Megasphaera elsdenii, Mitsuokella multiacida, Roseburia intestinalis, Faecalibacterium prausnitzii and Eubacterium hallii.

Within the scope of the present invention, butyric acid is of considerable interest due to its ability to give energetic support to colonocytes, maintain the intestinal mucosa intact through stimulation of mucus production, and inhibit the activity of pro-inflammatory mediators at the intestinal epithelium.

A double-blind, placebo-controlled, randomized clinical trial shows the effects of microencapsulated butyric acid on symptoms and quality of life of IBS patients. In the trial, 300 mg of microencapsulated butyric acid or placebo are administered per day as an adjunct to standard therapies. A significant decrease in the frequency of abdominal pain associated with defecation occurs during the fourth week of treatment in patients administered with butyric acid. Spontaneous abdominal pain, post-prandial abdominal pain, defecation-associated pain, and post-defecation stimulus also decrease during the twelfth week. Another clinical trial shows the effects of butyric acid on IBS-D patients. 50 patients, of whom 22 with IBS-D and 28 with IBS-C, receive 1 g/day of butyric acid (divided into 4 administrations with 250 mg capsules) and inulin for 1 month. Treatment with butyric acid results in a normalization of the condition of IBS-D patients, with a significant reduction in the frequency of diarrhea episodes and a significant increase in stool consistency compared to IBS-C patients.

Non-limiting examples of butyric acid salts suitable for use within the scope of the present invention are sodium butyrate or calcium butyrate.

Tamarind is a tropical fruit tree belonging to the Fabaceae family, which can reach 24 metres in height and 7 metres in width and has pale yellow and pink flowers. It needs a dry climate in order to grow, so it grows more in the areas of East Africa and India, but also in the tropical areas of Asia and Latin America. All the parts of the tamarind have not only a great nutritional value, but also wide use in medicine. According to the World Health Organization, the tamarind fruit is an ideal source of all essential amino acids, except tryptophan. Even its seeds have similar properties, therefore Tamarind is an important source of protein, especially in those areas where protein deficiency is a common problem. Chemical analysis results indicate that Tamarind contains phenolic compounds such as procyanidins, catechins, epicatechins, tartaric acid, mucilages, pectins, arabinose, xylose, galactose, uronic acid, and triterpenes. The seeds are rich in tannins, saponins, flavonoids, alkaloids, and glycosides, which account for the anti-inflammatory and analgesic effect of Tamarind, which also has an antimicrobial effect on pathogens such as Salmonella paratyphi, Bacillus subtilis, Salmonella typhi, and Staphylococcus aureus. The substances contained within the aforementioned active principle make this species linkable to the treatment of various disorders of the gastrointestinal system.

Tamarind is in fact used as a laxative, above all due to its content in malic acid, tartaric acid, and potassium. Tamarind seed extract was also shown to have a dose-dependent protective effect on ibuprofen-, alcohol- or pylorus ligation-induced ulcer models. This is due to the phenolic content, especially in procyanidins, epicatechins and polymeric tannins, which have an antioxidant action and prevent ulcer development through protein accumulation and vasoconstriction. Tamarind also exerts a spasmolytic action by blocking the calcium channels, resulting in relaxation of the intestinal smooth muscles, which can be useful in case of diarrhea. Within the scope of the present invention, Tamarind is interesting because it can bring beneficial effects in case of abdominal pain associated with diarrhea or constipation. In fact, in particular, the leaves (for diarrhea), the fruit (for constipation) and the soft parts of the bark and root (for abdominal pain in general), can be used to alleviate the typical manifestations of irritable bowel syndrome.

An in vivo study in rats shows the analgesic and anti-inflammatory effect of the methanolic extract of Tamarind seeds. Inflammation is induced by administering 0.1 ml of carrageenan in the sub-plantar area of the rats' left hind paw. The animals are divided into 5 groups:

1—Distilled water (10 ml/kg)+Carrageenan (0.1 ml in 1% saline);

2—Diclofenac sodium (10 mg/kg p.o.)+Carrageenan (0.1 ml in 1% saline);

3—Methanolic extract of Tamarindus indica (100 mg/kg p.o.)+Carrageenan (0.1 ml in 1% saline);

4—Methanolic extract of Tamarindus indica (200 mg/kg p.o.)+Carrageenan (0.1 ml in 1% saline);

5—Methanolic extract of Tamarindus indica (400 mg/kg p.o.)+Carrageenan (0.1 ml in 1% saline).

Carrageenan is administered to all animals 1 hour after treatment with Diclofenac or Tamarind, and the volume of the paw is measured plethysmometrically at 1 h, 2 h, 3 h, 4 h, 5 h, and 24 h. Diclofenac-treated rats showed a significant decrease in edema from 1 to 24 h (p<0.01) compared to the control. Rats treated with 100 mg/kg Tamarind showed a significant decrease at 5 h (p<0.05) and 24 h (p<0.01) compared to the control. Rats treated with 200 mg/kg Tamarind showed a significant decrease at 2 h (p<0.05) and from 3 h to 24 h (p<0.01) compared to the control. Rats treated with 400 mg/kg Tamarind showed a significant decrease at 1 h (p<0.05) and from 2 to 24 h (p<0.01) compared to the control. Therefore, the methanolic extract of Tamarind showed a dose-dependent percentage of inhibition of carrageenan-induced edema and resulted, especially when administered at 400 mg/kg, in recovery of the neutrophil and lymphocyte counts, which are often disrupted in the inflammatory process.

The analgesic activity is assessed within the same study through the tail immersion method. The animals are divided into 5 groups:

1—Distilled water (10 ml/kg)

2—Pentazocine (30 mg/kg);

3—Methanolic extract of Tamarindus indica (100 mg/kg p.o.);

4—Methanolic extract of Tamarindus indica (200 mg/kg p.o.);

5—Methanolic extract of Tamarindus indica (400 mg/kg p.o.);

After the respective drug treatment, the tail of each animal was immersed in a beaker of freshly filled water at exactly 55° C. and the reaction time (the rat's tail withdrawal) was measured at 0, 15, 30, 45 and 60 minutes, respectively. The Pentazocine-treated group showed a significant increase (p<0.01) in the reaction time at 15, 30, 45 and 60 minutes compared to the control group. The group treated with 100 mg/kg Tamarind showed a significant increase (p<0.05) in the reaction time at 45 minutes compared to the control group. The group treated with 200 mg/kg Tamarind showed a significant increase (p<0.01) in the reaction time at 30 and 45 minutes, as well as at 15 and 60 minutes (p<0.05) compared to the control group. The group treated with 400 mg/kg Tamarind showed a significant increase (p<0.01) in the reaction time at 15, 30, 45 and 60 minutes compared to the control group. Therefore, the methanolic extract of Tamarind showed a dose-dependent analgesic activity.

A randomized, multicenter clinical trial in children (from 3 months to 12 years of age) suffering from acute gastroenteritis shows the antidiarrheal effects of xyloglucans extracted from Tamarind seeds. The children are randomized into taking Tamarind together with a rehydration solution or only into taking the rehydration solution orally. Tamarind xyloglucans significantly reduced diarrheal episodes in children with gastroenteritis already six hours after intake. This action is due to the ability of xyloglucans to form a protective biofilm on the intestinal mucosa that improves the mucosal resistance to pathogen aggression and helps restore its normal function. A trial carried out in adults also showed the efficacy and safety of Tamarind xyloglucans in patients with diarrhea, with a quicker action than other antidiarrheal products such as the probiotic Saccharomyces boulardii and diosmectite, an aluminium and magnesium silicate used as an intestinal adsorbent in the most common forms of gastroenteritis. By virtue of the different stated properties, the various Tamarind extracts are of considerable interest in the field of prevention and treatment of IBS symptoms by acting on different characteristic aspects of the disease (pain, diarrhea, constipation).

As indicated above, the composition of the present invention is effective in the treatment and/or prevention of irritable bowel syndrome.

The present invention simultaneously allows:
An analgesic effect
An anti-inflammatory effect
An antioxidant effect
An effect in modulating intestinal transit

More particularly, the present invention provides a valid and prompt intervention tool for counteracting the typical symptoms of irritable bowel syndrome such as diarrhea, constipation and abdominal pain. Butyric acid is a valid energy source for intestinal epithelial cells and helps restore mucosal integrity; furthermore, this compound is capable of inhibiting the pro-inflammatory activity of cytokines at the intestinal epithelium, thus improving the various symptoms of IBS (constipation, diarrhea). Tamarind, thanks to its multiple components (flavonoids, saponins, tannins, glycosides) performs an anti-inflammatory, analgesic, antioxidant and antimicrobial action. Tamarind xyloglucans, in particular, form a film on the intestinal mucosa which allows the attack by the most common pathogenic microorganisms to be prevented. The fruits, on the other hand, thanks to their content of various metabolites, promote intestinal transit.

The efficacy of the composition object of the present invention has been assessed according to experimental protocols known to those skilled in the art. In particular, in vitro and/or in vivo assays known in the scientific literature can be used for assessing the different actions of the composition according to the present invention.

In vitro assays which assess the ability to inhibit the release of inflammatory cytokines, such as IL-1, IL-6 and TNF-α, and the expression of enzymes, such as COX-2 and IL-1β-induced metalloprotease-13, in primary human cell cultures (e.g. macrophages, chondrocytes, and fibroblasts), are suitable to demonstrate the anti-inflammatory efficacy of the composition according to the present invention, which is of particular interest for the treatment of irritable colon syndrome.

On the other hand, in vitro assays, such as, for example, the DPPH test, the radical scavenging activity on nitric oxide or on the peroxy-nitrile radical, the TEAC (Total radical-trapping antioxidant parameter), FRAP (Ferric reducing-antioxidant power), HORAC (Hydroxyl radical averting capacity), ORAC (Oxygen radical absorbance capacity) tests, and the like, are suitable to demonstrate the antioxidant efficacy of the composition according to the present invention, which is of particular interest for the prevention and/or treatment of IBS.

An in vivo model proposes to test the present invention for its anti-inflammatory effect in mice. After anesthesia, dinitrobenzensulphonic acid (DNBS) is injected into the colon, by rectal administration via a polyethylene catheter. DNBS injection causes strong mucosal tissue damage, granulocyte infiltration into the mucosa/submucosa with edema and inflammation. The purpose of the model is to demonstrate that oral administration of the present composition reduces the signs of damage to the colon, thus bringing about a significant reduction in edema and erosion areas in the mucosa and submucosa compared to a control group and to the individual components of the composition under examination. Furthermore, DNBS-induced inflammation causes a significant increase in myeloperoxidase (MPO) activity, which is considered as an index of neutrophil infiltration and a parameter to quantify intestinal inflammation. The experimental model proposes to demonstrate that the activity of these enzymes is significantly reduced by oral administration of Tamarind and Butyric acid to a greater extent than with the control and the intake of the individual components. Moreover, the composition under examination intends to enhance intestinal permeability, which is significantly disrupted by intracolonic administration of dinitrobenzensulphonic acid.

Chronic stress models such as the WRS (Wrap Restrain Stress), MS (Maternal Separation) and WAS (Water avoidance Stress) tests can also be used as in vivo models to induce typical IBS symptoms in animals. After establishing the experimental model, the animals are administered with the substances either individually or in combination in order to confirm their synergistic action.

The synergistic action of the active ingredients of the present invention is also assessed in relation to their ability to modulate intestinal transit. Specifically, experimental in vivo protocols can be used to demonstrate the pro-kinetic and laxative effect useful in case of constipation; and the antidiarrheal effect useful in the case of irritable bowel syndrome in the diarrhea predominant phase.

Experimental models that can be used are all those known to the person skilled in the art such as a model of loperamide-induced constipation, intestinal transit assessment using activated charcoal, diarrhea induced by magnesium sulphate, serotonin, castor oil.

In a preferred embodiment, the composition of the present invention is prepared as a pharmaceutical dosage form comprising from 1 mg to 10 g of butyric acid, or a salt thereof, and from 1 mg to 10 g of Tamarindus indica extract, in addition to the usual excipients used in the preparation of the selected dosage form.

Suitable amounts of butyric acid are 2 mg, 3 mg, 4 mg, 5 mg, 6 mg, 7 mg, 8 mg, 9 mg, 10 mg, 20 mg, 30 mg, 40 mg, 50 mg, 60 mg, 70 mg, 80 mg, 90 mg, 100 mg, 110 mg, 120 mg, 130 mg, 140 mg, 150 mg, 160 mg, 170 mg, 180 mg, 190 mg, 200 mg, 210 mg, 220 mg, 230 mg, 240 mg, 250 mg, 260 mg, 270 mg, 280 mg, 290 mg, 300 mg, 310 mg, 320 mg, 330 mg, 340 mg, 350 mg, 360 mg, 370 mg, 380 mg, 390 mg, 400 mg, 410 mg, 420 mg, 430 mg, 440 mg, 450 mg, 460 mg, 470 mg, 480 mg, 490 mg, 500 mg, 510 mg, 520 mg, 530 mg, 540 mg, 550 mg, 560 mg, 570 mg, 580 mg, 590 mg, 600 mg, 610 mg, 610 mg, 620 mg, 630 mg, 640 mg, 650 mg, 660 mg, 670 mg, 680 mg, 690 mg, 700 mg, 710 mg, 720 mg, 730 mg, 740 mg, 750 mg, 760 mg, 770 mg, 780 mg, 790 mg, 800 mg, 810 mg, 820 mg, 830 mg, 840 mg, 850 mg, 860 mg, 870 mg, 880 mg, 890 mg, 900 mg, 910 mg, 920 mg, 930 mg, 940 mg, 950 mg, 960 mg, 970 mg, 980 mg, 990 mg, 1000 mg (1 g), 2 g, 3 g, 4 g, 5 g, 6 g, 7 g, 8 g, 9 g, 10.

Suitable amounts of Tamarindus indica extract are 2 mg, 3 mg, 4 mg, 5 mg, 6 mg, 7 mg, 8 mg, 9 mg, 10 mg, 20 mg, 30 mg, 40 mg, 50 mg, 60 mg, 70 mg, 80 mg, 90 mg, 100 mg, 110 mg, 120 mg, 130 mg, 140 mg, 150 mg, 160 mg, 170 mg, 180 mg, 190 mg, 200 mg, 210 mg, 220 mg, 230 mg, 240 mg, 250 mg, 260 mg, 270 mg, 280 mg, 290 mg, 300 mg, 310 mg, 320 mg, 330 mg, 340 mg, 350 mg, 360 mg, 370 mg, 380 mg, 390 mg, 400 mg, 410 mg, 420 mg, 430 mg, 440 mg, 450 mg, 460 mg, 470 mg, 480 mg, 490 mg, 500 mg, 510 mg, 520 mg, 530 mg, 540 mg, 550 mg, 560 mg, 570 mg, 580 mg, 590 mg, 600 mg, 610 mg, 610 mg, 620 mg, 630 mg, 640 mg, 650 mg, 660 mg, 670 mg, 680 mg, 690 mg, 700 mg, 710 mg, 720 mg, 730 mg, 740 mg, 750 mg, 760 mg, 770 mg, 780 mg, 790 mg, 800 mg, 810 mg, 820 mg, 830 mg, 840 mg, 850 mg, 860 mg, 870 mg, 880 mg, 890 mg, 900 mg, 910 mg, 920 mg, 930 mg, 940 mg, 950 mg, 960 mg, 970 mg, 980 mg, 990 mg, 1000 mg (1 g), 2 g, 3 g, 4 g, 5 g, 6 g, 7 g, 8 g, 9 g, 10 g.

The present description further includes any range of values between two of the aforementioned quantities, both with reference to butyric acid and with reference to Tamarindus indica extract.

Furthermore, all the quantities and ranges described herein with reference to butyric acid and Tamarindus indica extract can be combined with each other.

Dosage regimen: the dosage indicated above refers to the content by weight of the substances per single dosage unit.

In a further preferred embodiment, the composition of the present invention is prepared as a pharmaceutical dosage form comprising:
from 0.1 to 90% by weight of butyric acid, or a salt thereof, preferably from 1% to 50% by weight; and
from 0.1 to 90% by weight of Tamarindus indica extract, preferably from 1% to 50% by weight;

in addition to the usual excipients used in the preparation of the selected dosage form. The percentages by weight indicated above refer to the total weight of the composition including any excipients, carriers and/or diluents.

Additional butyric acid concentration values suitable for use in the composition of the invention are 0.2, 0.3, 0.4, 0.5, 0.6, 0.7, 0.8, 0.9, 1, 1.5, 2, 2.5, 3, 3.5, 4, 4.5, 5. 5.5, 6, 6.5, 7, 7.5, 8, 8.5, 9, 9.5, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90%.

Additional Tamarindus indica extract concentration values suitable for use in the composition of the invention are 0.2, 0.3, 0.4, 0.5, 0.6, 0.7, 0.8, 0.9. 1, 1.5, 2, 2.5, 3, 3.5, 4, 4.5, 5, 5.5, 6, 6.5, 7, 7.5, 8, 8.5, 9, 9.5, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90%.

The present description further includes any range of values between two of the aforementioned concentrations, both with reference to butyric acid and with reference to Tamarindus indica extract.

Furthermore, all the quantities and ranges described herein with reference to butyric acid and Tamarindus indica extract concentrations can be combined with each other.

The composition of the present invention is preferably administered orally. For this purpose, the preferred pharmaceutical dosage forms are tablets, coated tablets, modified-release tablets, capsules, modified-release capsules, gastro-resistant capsules, soft capsules, powders, granulates, solutions, suspensions, syrups.

The following examples are provided for illustration purposes only and are not intended to limit the scope of the invention as defined in the appended claims.

### EXAMPLES

### Example 1

| | Amount |
|---|---|
| Active ingredient | per capsule |
| Butyric acid | 400 mg |
| Tamarind | 150 mg |

Pharmaceutical form: capsules

### Example 2

| | Amount |
|---|---|
| Active ingredient | per capsule |
| Butyric acid | 300 mg |
| Tamarind | 100 mg |

Pharmaceutical form: capsules

### Example 3

| | Amount |
|---|---|
| Active ingredient | per tablet |
| Butyric acid | 250 mg |
| Tamarind | 200 mg |

Pharmaceutical form: tablets

### Example 3

| | Amount per |
|---|---|
| Active ingredient | measuring scoop |
| Butyric acid | 250 mg |
| Tamarind | 100 mg |

Pharmaceutical form: oral bottle

### Example 4

| | Amount | |
|---|---|---|
| Active ingredient | per sachet | |
| Butyric acid | 200 | mg |
| Tamarind | 50 | mg |

Pharmaceutical form: sachets

What is claimed is:

1. A composition comprising butyric acid or a salt thereof and Tamarindus indica extract.

2. The composition of claim 1, wherein the butyric acid salt is selected from the group consisting of calcium butyrate and sodium butyrate.

3. The composition of claim 1, wherein the composition is a pharmaceutical dosage form for oral administration.

4. The composition of claim 3, wherein the composition is a pharmaceutical dosage form selected from the group consisting of tablets, coated tablets, modified-release tablets, capsules, modified-release capsules, gastro-resistant capsules, soft capsules, powders, granulates, solutions, suspensions, and syrups.

5. The composition of claim 1, comprising from 1 mg to 10 g of the butyric acid or the salt thereof and from 1 mg to 10 g of the Tamarindus indica extract per unit dose.

6. The composition of claim 1, comprising from 0.1 to 90% by weight of the butyric acid or the salt thereof and from 0.1 to 90% by weight of the Tamarindus indica extract, the percentages being referred to the total weight of the composition optionally including excipients, carriers, and/or diluents.

7. The composition of claim 6, comprising from 1 to 50% by weight of the butyric acid or the salt thereof and from 1 to 50% by weight of the Tamarindus indica extract.

8. A method for the treatment and/or prevention of irritable bowel syndrome, said method comprising administering to a subject in need thereof the composition of claim 1.

9. The method of claim 8, wherein the treatment is a symptomatic therapeutic treatment.
